(19) United States
(12) Patent Application Publication
(10) Pub. No.: US 2023/0098301 A1
(43) Pub. Date: Mar. 30, 2023

(54) BIOMETRIC MEASUREMENT ADJUSTMENT FOR ACTIVITY

(71) Applicant: Lenovo (United States) Inc., Morrisville, NC (US)

(72) Inventors: Justin Michael Ringuette, Morrisville, NC (US); Sandy Collins, Durham, NC (US); Mark K Summerville, Apex, NC (US); Mark Christopher Heidenfeldt, Raleigh, NC (US); Aravind Senthil Murugan, Morrisville, NC (US)

(21) Appl. No.: 17/490,438

(22) Filed: Sep. 30, 2021

(51) Int. Cl. A63B 24/00; G16H 20/30; G16H 50/30; G16H 40/67

(52) U.S. Cl. CPC A63B 24/0062; G16H 20/30; G16H 50/30; G16H 40/67; G16H 10/60

(57) ABSTRACT

One embodiment provides a method, including: identifying, using an information handling device, an activity engaged in by a user; determining, using a processor, whether a biometric of the user during the activity is affected by a biometric-affecting aspect; and adjusting, responsive to determining that the biometric is affected by the biometric-affecting aspect, an estimated measurement for the biometric. Other aspects are described and claimed.

### BACKGROUND

Individuals utilize a variety of different types of information handling devices (“devices”), for example smart phones, wearable devices, purpose-built health trackers, and the like, to track and record various types of fitness and/or biometric data. For example, many individuals may be interested in knowing how many steps they took during a particular time period (e.g., over the course of a day, during an event, etc.) or how many calories were burned during a particular activity (e.g., during a workout, etc.).

### BRIEF SUMMARY

In summary, one aspect provides a method, including: identifying, using an information handling device, an activity engaged in by a user; determining, using a processor, whether a biometric of the user during the activity is affected by a biometric-affecting aspect; and adjusting, responsive to determining that the biometric is affected by the biometric-affecting aspect, an estimated measurement for the biometric.

Another aspect provides an information handling device, including: a processor; a memory device that stores instructions executable by the processor to: identify an activity engaged in by a user; determine whether a biometric of the user during the activity is affected by a biometric-affecting aspect; and adjust, responsive to determining that the biometric is affected by the biometric-affecting aspect, an estimated measurement for the biometric.

A further aspect provides a product, including: a storage device that stores code, the code being executable by a processor and comprising: code that identifies an activity engaged in by a user; code that determines whether a biometric of the user during the activity is affected by a biometric-affecting aspect; and code that adjusts, responsive to determining that the biometric is affected by the biometric-affecting aspect, an estimated measurement for the biometric.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1 illustrates an example of information handling device circuitry.

FIG. 2 illustrates another example of information handling device circuitry.

FIG. 3 illustrates an example method of adjusting an estimated measurement for a biometric of a user during engagement in an activity.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

Over the last several years, an increased emphasis on health and well-being has led to the prevalence of fitness tracking devices in our society. These accessories are able to track and record various types of user biometric data (e.g., heartrate, step count, calories burned, etc.) that users may rely on to track their progress toward their health or fitness goals and/or to maintain healthy habits. Conventionally, these biometrics are estimated via an algorithm that utilizes data obtained by one or more sensors (e.g., a pedometers, accelerometers, heart rate sensors, etc.). The accuracy of the estimates may be further increased if additional user-specific information (e.g., a user's age, gender, height, weight, etc.) is provided to the fitness tracking device or application (e.g., by the user, etc.).

Although the biometric estimates described above may be generally reliable for many individuals, the same cannot be said for those individuals who have disabilities and/or who engage in activities using various types of assistive devices. For example, a disabled user may expend more calories performing the same activity as an able-bodied user. As another example, a user performing an activity with an assistive device (e.g., an assistive workout machine, a limb prosthetic, etc.) may expend fewer calories performing that activity than a user without the assistive device.

Solutions exist that may be employed to address the foregoing issues. However, these solutions are all manual in nature and may be burdensome for a user to implement. For example, users may manually edit their biometrics after each tracked activity (e.g., a user can edit the estimated calories burned for an activity, etc.). In addition to being highly labor-intensive, such a solution may invite inaccuracies because users largely increase or decrease a value for a biometric based on their best-guess for how their contextual situation may affect the biometric value for the biometric (e.g., a disabled user may increase a burned calorie estimate for an activity by an arbitrary amount, etc.).

Accordingly, an embodiment provides a method for dynamically adjusting an estimated measurement for a biometric based on a determined biometric-affecting aspect associated with a user. In an embodiment, an indication to activate a fitness tracking application may be received at a device. An embodiment may then identify an activity engaged in by a user and determine whether a biometric of the user affected by a biometric-affecting aspect (e.g., a disability native to the user, a prosthetic worn the user, an assistive device utilized by the user, etc.). Responsive to determining that a user's biometrics were affected by the biometric-affecting aspect, an estimated measurement for the biometric may be dynamically adjusted. Such a method may provide a more accurate accounting of a user's biometrics during an activity.

The illustrated example embodiments will be best understood by reference to the figures. The following description is intended only by way of example, and simply illustrates certain example embodiments.

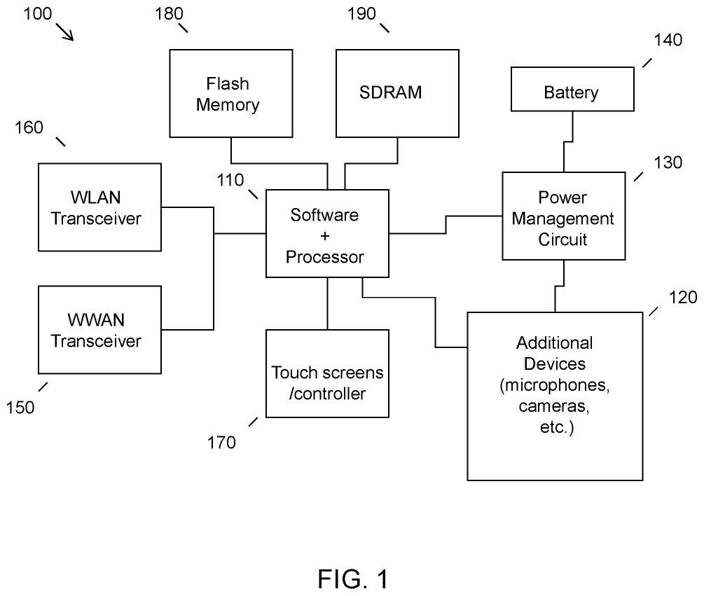

While various other circuits, circuitry or components may be utilized in information handling devices, with regard to smart phone and/or tablet circuitry 100, an example illustrated in FIG. 1 includes a system on a chip design found for example in tablet or other mobile computing platforms. Software and processor(s) are combined in a single chip 110. Processors comprise internal arithmetic units, registers, cache memory, busses, I/O ports, etc., as is well known in the art. Internal busses and the like depend on different vendors, but essentially all the peripheral devices (120) may attach to a single chip 110. The circuitry 100 combines the processor, memory control, and I/O controller hub all into a single chip 110. Also, systems 100 of this type do not typically use SATA or PCI or LPC. Common interfaces, for example, include SDIO and I2C.

There are power management chip(s) 130, e.g., a battery management unit, BMU, which manage power as supplied, for example, via a rechargeable battery 140, which may be recharged by a connection to a power source (not shown). In at least one design, a single chip, such as 110, is used to supply BIOS like functionality and DRAM memory.

System 100 typically includes one or more of a WWAN transceiver 150 and a WLAN transceiver 160 for connecting to various networks, such as telecommunications networks and wireless Internet devices, e.g., access points. Additionally, devices 120 are commonly included, e.g., an image sensor such as a camera, audio capture device such as a microphone, etc. System 100 often includes one or more touch screens 170 for data input and display/rendering. System 100 also typically includes various memory devices, for example flash memory 180 and SDRAM 190.

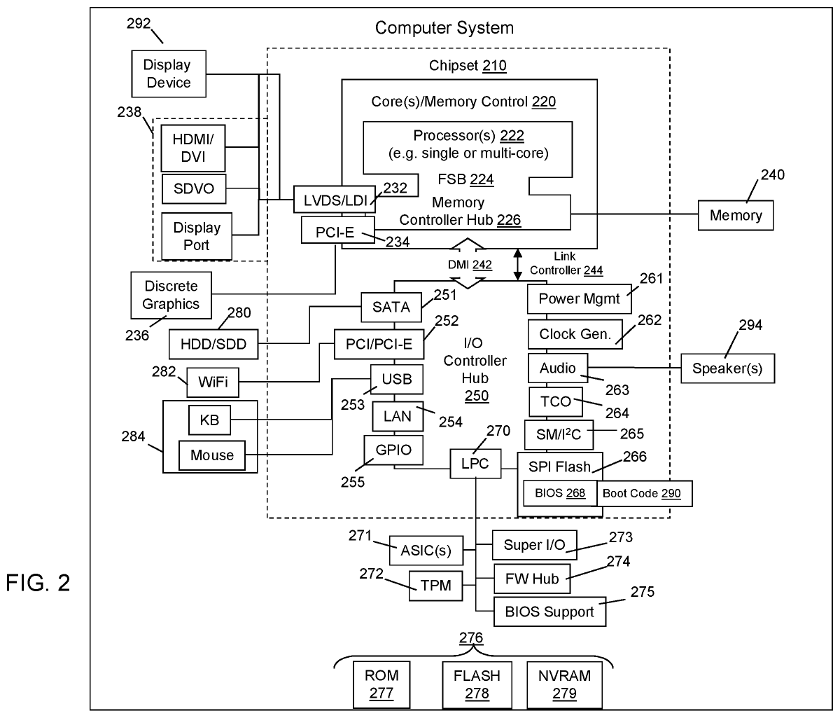

FIG. 2 depicts a block diagram of another example of information handling device circuits, circuitry or components. The example depicted in FIG. 2 may correspond to computing systems such as the THINKPAD series of personal computers sold by Lenovo (US) Inc. of Morrisville, N.C., or other devices. As is apparent from the description herein, embodiments may include other features or only some of the features of the example illustrated in FIG. 2.

The example of FIG. 2 includes a so-called chipset 210 (a group of integrated circuits, or chips, that work together, chipsets) with an architecture that may vary depending on manufacturer (for example, INTEL, AMD, ARM, etc.). INTEL is a registered trademark of Intel Corporation in the United States and other countries. AMD is a registered trademark of Advanced Micro Devices, Inc. in the United States and other countries. ARM is an unregistered trademark of ARM Holdings plc in the United States and other countries. The architecture of the chipset 210 includes a core and memory control group 220 and an I/O controller hub 250 that exchanges information (for example, data, signals, commands, etc.) via a direct management interface (DMI) 242 or a link controller 244. In FIG. 2, the DMI 242 is a chip-to-chip interface (sometimes referred to as being a link between a “northbridge” and a “southbridge”). The core and memory control group 220 include one or more processors 222 (for example, single or multi-core) and a memory controller hub 226 that exchange information via a front side bus (FSB) 224; noting that components of the group 220 may be integrated in a chip that supplants the conventional “northbridge” style architecture. One or more processors 222 comprise internal arithmetic units, registers, cache memory, busses, I/O ports, etc., as is well known in the art.

In FIG. 2, the memory controller hub 226 interfaces with memory 240 (for example, to provide support for a type of RAM that may be referred to as “system memory” or “memory”). The memory controller hub 226 further includes a low voltage differential signaling (LVDS) interface 232 for a display device 292 (for example, a CRT, a flat panel, touch screen, etc.). A block 238 includes some technologies that may be supported via the LVDS interface 232 (for example, serial digital video, HDMI/DVI, display port). The memory controller hub 226 also includes a PCI-express interface (PCI-E) 234 that may support discrete graphics 236.

In FIG. 2, the I/O hub controller 250 includes a SATA interface 251 (for example, for HDDs, SDDs, etc., 280), a PCI-E interface 252 (for example, for wireless connections 282), a USB interface 253 (for example, for devices 284 such as a digitizer, keyboard, mice, cameras, phones, microphones, storage, other connected devices, etc.), a network interface 254 (for example, LAN), a GPIO interface 255, a LPC interface 270 (for ASICs 271, a TPM 272, a super I/O 273, a firmware hub 274, BIOS support 275 as well as various types of memory 276 such as ROM 277, Flash 278, and NVRAM 279), a power management interface 261, a clock generator interface 262, an audio interface 263 (for example, for speakers 294), a TCO interface 264, a system management bus interface 265, and SPI Flash 266, which can include BIOS 268 and boot code 290. The I/O hub controller 250 may include gigabit Ethernet support.

The system, upon power on, may be configured to execute boot code 290 for the BIOS 268, as stored within the SPI Flash 266, and thereafter processes data under the control of one or more operating systems and application software (for example, stored in system memory 240). An operating system may be stored in any of a variety of locations and accessed, for example, according to instructions of the BIOS 268. As described herein, a device may include fewer or more features than shown in the system of FIG. 2.

Information handling circuitry, as for example outlined in FIG. 1 or FIG. 2, may be used in devices that are capable of detecting and/or recording user biometric data. For example, the circuitry outlined in FIG. 1 may be implemented in a wearable device, whereas the circuitry outlined in FIG. 2 may be implemented in a mobile computing device.

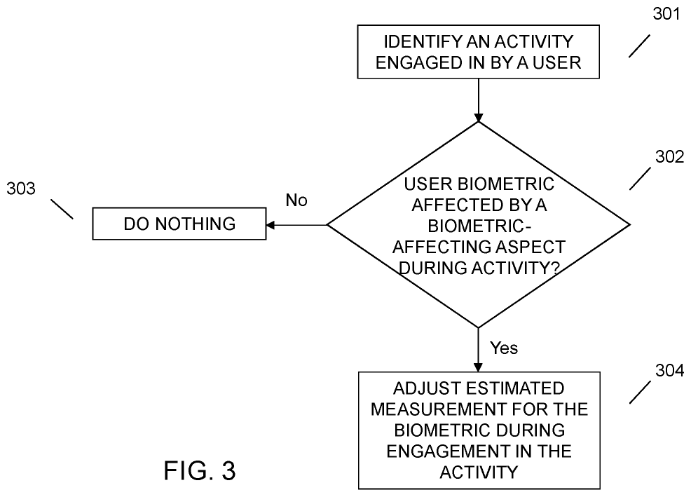

Referring now to FIG. 3, an embodiment provides a method of dynamically adjusting an estimated measurement for a biometric based upon a determined biometric-affecting aspect. At 301, an embodiment may identify an activity engaged in by a user. In the context of this application, the activity may be virtually any type of physical activity. More particularly, the activity may be a designated physical activity (e.g., a specific exercise type, a particular game or sporting match, etc.) or a passive physical activity (e.g., walking, etc.).

The identification of the activity may be facilitated automatically (i.e., without additional and explicit user input) using some or all of the following activity identification techniques. In one embodiment, a system may have access to a user's calendar data that may specify the type of activity the user will be engaged in at a particular time of day (e.g., an embodiment may identify that a user has a tennis match scheduled at noon, etc.). In another embodiment, a system having the appropriate permissions may access a user's social media and/or communication data (e.g., emails, text messages, voicemails, etc.) to identify the activity. For example, an embodiment may identify an agreement a user has made with their friend over text message to engage in a workout at a particular time in the afternoon. In yet another embodiment, data obtained from one or more sensors (e.g., camera sensors, accelerometers, gyroscopes, etc.) integrally or operatively coupled to the user's device may be utilized to identify the activity. For example, accelerometer data may indicate that a user is walking or running.

At 302, an embodiment may determine whether a biometric of a user is affected by a biometric-affecting aspect during engagement in the identified activity. In the context of this application, a biometric may be virtually any fitness-related metric that is objectively measurable. Non-limiting examples of possible biometrics may include calories burned during an activity, a user's heartrate during an activity, maximum rate of oxygen consumption (i.e., VO₂ max) during an activity, and the like. Furthermore, in the context of this application, a biometric-affecting aspect may refer to a characteristic of a user, or to a specific object, that may modulate (i.e., increase or decrease) the difficulty of an activity. Non-limiting examples of potential biometric-affecting aspects include one or more physical impediments inherent to the user (e.g., a missing limb, a low or non-functioning appendage, another type of disability that impedes natural movement in some way, etc.) or one or more assistive devices (e.g., a prosthetic, a support object such as a wheelchair or cane, a difficulty modulating object such as a weighted vest or a machine having weight resistance aspects, etc.) utilized by the user during the activity.

An embodiment may identify that a biometric-affecting aspect is present using some or all of the following biometric-affecting aspect identification techniques. For example, an embodiment may have access to a dedicated user fitness profile that contains information (e.g., entered by the user, etc.) regarding the biometric-affecting aspects that may be present in different activities. Additionally or alternatively, an embodiment may dynamically identify the biometric-affecting aspect present via data obtained from available sensors (e.g., camera sensors, audio capture devices, etc.) and/or from context data obtained from various accessible sources (e.g., social media data, communication data, hospital record data if available, etc.). For example, an embodiment having access to a user's social media may identify a recent post in which the user stated, “just got my new prosthetic leg, can't wait to test it out this afternoon on my run!”

In an embodiment, the determination that a biometric is affected by a biometric affecting-aspect may be solely based on the identification that a biometric-affecting aspect is present. Stated differently, responsive to identifying that a biometric-affecting aspect is present during the activity, an embodiment may automatically conclude that one or more biometrics are being affected by it. For example, responsive to identifying that a user has a missing appendage, an embodiment may automatically conclude that a biometric for any activity will be affected by this physical impediment.

Alternatively to the foregoing, this determination may be more context-driven by identifying a nature of the biometric-affecting aspect and its specific involvement in the activity. For example, an embodiment may initially identify that a user is outfitted with an above-knee prosthetic. An embodiment may thereafter identify that such a support device would significantly affect the calories burned while the user was engaged in one activity (e.g., walking, running, etc.) but may not significantly affect the calories burned while the user was engaged in another activity (e.g., boxing, weight-lifting, etc.). Accordingly, an embodiment may have access to a database (e.g., stored locally on the device, stored remotely on another device or server, etc.) that contains difficulty modulating information for each activity a specific biometric-affecting aspect may be involved in. Responsive to identifying that the specific biometric-affecting aspect modulates the difficulty of an activity by some predetermined threshold amount, an embodiment may conclude that the relevant biometric is affected by the biometric-affecting aspect. If an embodiment identifies that the specific biometric-affecting aspect does not modulate the difficulty of the activity by at least the predetermined threshold amount, an embodiment may conclude that the affect the biometric-affecting aspect has on the activity is not significant. In an embodiment, the predetermined threshold may vary between different biometrics, different activities, and different biometric-affecting aspects.

Responsive to determining, at 302, that a biometric of the user is unaffected by a biometric-affecting aspect and/or that no biometric-affecting aspect is present, and embodiment may, at 303, take no additional action. Stated differently, an embodiment may measure and record the biometric using one or more conventional means. Conversely, responsive to determining, at 302, that a biometric of the user is affected by a biometric-affecting aspect during engagement in the activity, an embodiment may, at 304, adjust an estimated measurement for the biometric.

In an embodiment, the adjustment of the estimated measurement may occur automatically and without receipt of any additional user input. Additionally, in an embodiment, the amount by which the estimated measurement is adjusted may be deduced by access to one or more available data sources (e.g., an available database, crowdsourced data, etc.). These data sources may contain information that identifies by what value or by what factor an estimate for a particular biometric may be affected by a specific biometric-affecting aspect. For example, an accessible database may contain calories burned information for walking with a leg prosthetic. This information may subsequently be incorporated into an existing calorie-counting algorithm.

In an embodiment, the adjustment may correspond to an increase in the value of the biometric. As a non-limiting example, a user may have some type of physical impediment (e.g., a missing limb, a low or non-functioning appendage, etc.) that makes engagement in an activity more strenuous than for able-bodied individuals. Responsive to identifying that a user is engaging in the activity with this impediment, an embodiment may dynamically increase a calories-burned biometric for the activity by some predetermined amount. It is important to note that increases to the biometric estimates are not solely limited to users with inherent challenges, but rather, these estimate increases may also apply to users who willingly increase the difficulty of an activity by utilizing supplemental means. For example, a user may utilize a difficulty-modulating object (e.g., a weighted vest, a weight-resistance machine, a naturally occurring object such as a hill, etc.) to perform a task (e.g., a walk, a run, an exercise routine, etc.).

In an embodiment, the adjustment may correspond to a decrease in the value of the biometric. As a non-limiting example, a user may be engaged in a pull-up exercise on a machine at the gym. The pull-up machine may contain a pad that the user may rest their knees on while performing the pull-up. A user may adjust the weighted support the pad provides to the user during the workout by adding or removing weight that affects the pad. By increasing the support provided by the pad, the user may correspondingly decrease the difficulty of the exercise and thereby enable themselves to perform more pullups than they otherwise could unassisted. An embodiment may recognize the assistance provided by this support and thereafter dynamically decrease a calories burned biometric associated with this exercise.

The various embodiments described herein thus represent a technical improvement to conventional methods for tracking user biometrics during activities. Using the techniques described herein, an embodiment may identify an activity engaged in by a user. An embodiment may then determine whether a biometric of the user is affected during the activity by a biometric-affecting aspect. Responsive to determining that it is, an embodiment may dynamically adjust an estimate associated with the biometric. Such a method may provide more contextually accurate biometric estimates for different activities.

As will be appreciated by one skilled in the art, various aspects may be embodied as a system, method or device program product. Accordingly, aspects may take the form of an entirely hardware embodiment or an embodiment including software that may all generally be referred to herein as a “circuit,” “module” or “system.” Furthermore, aspects may take the form of a device program product embodied in one or more device readable medium(s) having device readable program code embodied therewith.

It should be noted that the various functions described herein may be implemented using instructions stored on a device readable storage medium such as a non-signal storage device that are executed by a processor. A storage device may be, for example, a system, apparatus, or device (e.g., an electronic, magnetic, optical, electromagnetic, infrared, or semiconductor system, apparatus, or device) or any suitable combination of the foregoing. More specific examples of a storage device/medium include the following: a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a storage device is not a signal and “non-transitory” includes all media except signal media.

Program code embodied on a storage medium may be transmitted using any appropriate medium, including but not limited to wireless, wireline, optical fiber cable, RF, et cetera, or any suitable combination of the foregoing.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), or the connection may be made through other devices (for example, through the Internet using an Internet Service Provider), through wireless connections, e.g., near-field communication, or through a hard wire connection, such as over a USB connection.

Example embodiments are described herein with reference to the figures, which illustrate example methods, devices and program products according to various example embodiments. It will be understood that the actions and functionality may be implemented at least in part by program instructions. These program instructions may be provided to a processor of a device, a special purpose information handling device, or other programmable data processing device to produce a machine, such that the instructions, which execute via a processor of the device implement the functions/acts specified.

It is worth noting that while specific blocks are used in the figures, and a particular ordering of blocks has been illustrated, these are non-limiting examples. In certain contexts, two or more blocks may be combined, a block may be split into two or more blocks, or certain blocks may be re-ordered or re-organized as appropriate, as the explicit illustrated examples are used only for descriptive purposes and are not to be construed as limiting.

As used herein, the singular “a” and “an” may be construed as including the plural “one or more” unless clearly indicated otherwise.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A method, comprising:
identifying, using an information handling device, an activity engaged in by a user;
determining, using a processor, whether a biometric of the user during the activity is affected by a biometric-affecting aspect; and
adjusting, responsive to determining that the biometric is affected by the biometric-affecting aspect, an estimated measurement for the biometric.

2. The method of claim 1, wherein the identifying the activity comprises automatically identifying the activity, without additional user input, by utilizing data obtained from at least one of: context data and a sensor.

3. The method of claim 1, wherein the biometric corresponds to a fitness metric selected from the group consisting of: a calorie count, a heart rate, and an oxygen consumption rate.

4. The method of claim 1, wherein the determining comprises:
identifying a way that the biometric-affecting aspect is utilized in the activity; and
determining whether the way that the biometric-affecting aspect is utilized in the activity affects the biometric by greater than a threshold amount

5. The method of claim 4, wherein the adjusting comprises adjusting responsive to determining that the biometric is affected by greater than the threshold amount.

6. The method of claim 1, wherein the biometric-affecting aspect is a physical impediment native to the user.

7. The method of claim 4, wherein the adjusting comprises increasing, in consideration of the physical impediment, the estimated measurement for the biometric during engagement in the activity.

8. The method of claim 1, wherein the biometric-affecting aspect is an assistive device for performance of the activity.

9. The method of claim 8, wherein the assistive device is selected from the group consisting of: a prosthetic, a support object, and a difficulty-modulating object.

10. The method of claim 8, wherein the adjusting comprises decreasing, in consideration of the assistive device, the estimated measurement of the biometric during engagement in the activity.

11. An information handling device, comprising:
a processor;
a memory device that stores instructions executable by the processor to:
identify an activity engaged in by a user;
determine whether a biometric of the user during the activity is affected by a biometric-affecting aspect; and
adjust, responsive to determining that the biometric is affected by the biometric-affecting aspect, an estimated measurement for the biometric.

12. The information handling device of claim 11, wherein the instructions executable by the processor to identify the activity comprise instructions executable by the processor to automatically identify the activity, without additional user input, by utilizing data obtained from at least one of: context data and a sensor.

13. The information handling device of claim 11, wherein the biometric corresponds to a fitness metric selected from the group consisting of: a calorie count, a heart rate, and an oxygen consumption rate.

14. The information handling device of claim 11, wherein the instructions executable by the processor to determine comprise instructions executable by the processor to:
identify a way that the biometric-affecting aspect is utilized in the activity; and
determine whether the way that the biometric-affecting aspect is utilized in the activity affects the biometric by greater than a threshold amount

15. The information handling device of claim 14, wherein the instructions executable by the processor to adjust comprise instructions executable by the processor to adjust responsive to determining that the biometric is affected by greater than the threshold amount.

16. The information handling device of claim 11, wherein the biometric-affecting aspect is a physical impediment native to the user.

17. The information handling device of claim 16, wherein the instructions executable by the processor to adjust comprise instructions executable by the processor to increase, in consideration of the physical impediment, the estimated measurement for the biometric during engagement in the activity.

18. The information handling device of claim 11, wherein the biometric-affecting aspect is an assistive device for performance of the activity, wherein the assistive device is selected from the group consisting of: a prosthetic, a support object, and a difficulty-modulating object.

19. The information handling device of claim 18, wherein the adjusting comprises decreasing, in consideration of the assistive device, the estimated measurement of the biometric during engagement in the activity.

20. A product, comprising:
a storage device that stores code, the code being executable by a processor and comprising:
code that identifies an activity engaged in by a user;
code that determines whether a biometric of the user during the activity is affected by a biometric-affecting aspect; and
code that adjusts, responsive to determining that the biometric is affected by the biometric-affecting aspect, an estimated measurement for the biometric.
